(19) United States
(12) Patent Application Publication
(10) Pub. No.: US 2013/0072565 A1
(43) Pub. Date: Mar. 21, 2013

(54) METHOD OF INCREASING CELL PROLIFERATION BY ADVANCED WOUND DRESSINGS

(71) Applicants: Jan G. Smith, Askim (SE); Mattias Andrup, Kullavik (SE)

(72) Inventors: Jan G. Smith, Askim (SE); Mattias Andrup, Kullavik (SE)

(21) Appl. No.: 13/623,564

(22) Filed: Sep. 20, 2012

Related U.S. Application Data
(60) Provisional application No. 61/626,136, filed on Sep. 21, 2011.

(51) Int. Cl. A61K 31/16; A61K 31/12; A61P 17/02; C12N 5/02

(52) U.S. Cl. 514/628; 435/375; 514/675

(57) ABSTRACT

The present invention relates generally to a method for stimulating cell proliferation. More specifically this invention discloses the use of increased cell proliferation in treatment of certain wounds.

### CROSS-REFERENCE TO RELATED APPLICATIONS

This application claims priority from U.S. Provisional Application Ser. No. 61/626,136 filed Sep. 21, 2011.

### BACKGROUND OF THE INVENTION

### Field of the Invention

The present invention relates generally to a method to stimulate cell proliferation in a wound. More specifically this invention relates to wound healing, due to this increased cell proliferation.

Wound healing is an intricate process in which the skin (or another organ) repairs itself after injury. Healing, like all other biological processes, is a cellular process. The occurrence of a wound immediately triggers the onset of this process, which continues until the injury is healed. As used herein, the term “wound” refers to tissue damage or loss of any kind, including but not limited to, cuts, incisions (including surgical incisions), abrasions, lacerations, contusions, bums, amputations and the like. Rapid healing of a wound reduces long term healthcare costs and improves patient recovery, including, regaining of sensation, function and aesthetics.

### SUMMARY OF THE INVENTION

It is an object of the present invention to provide the use of hydrophobic substances, e.g. dialkyl carbamoyl chloride or alkyl ketene dimer that preferably could be associated with a carrier to increase cell proliferation, which is ideal for treatment of wounds. This invention is based on the unexpected findings that hydrophobic substances including but not limited to dialkyl carbamoyl chloride and alkyl ketene dimer stimulate cell proliferation.

The present invention discloses the use of hydrophobic substances to induce cell proliferation in wounds.

The use of hydrophobic substances inducing cell proliferation is ideal for treatment of wounds, especially wounds with delayed healing process due to reduced cell proliferation and wounds where a quick healing process is desirable.

Other objects and features of the invention will be more fully apparent from the following disclosure and appended claims.

### BRIEF DESCRIPTION OF THE DRAWINGS

FIG. 1 shows the enhanced cell proliferation for fibroblasts associated with a carbamoyl chloride treated fabric compared to untreated control cells. (p=0,028: n=8),

FIG. 2 shows the BWAT score on day 1, 5, 10 and 15 for patients treated with dialkyl carbamoyl chloride treated wound dressings for enhanced cell and for patient treated with untreated wound dressings.

FIG. 3 shows the restoration of damaged fibroblast surface. By 72 hours, the restoration differed significantly between the dialkyl carbamoyl chloride group and untreated medium control (p=0.04331; n=6).

### DETAILED DESCRIPTION OF TUE INVENTION AND PREFERRED EMBODIMENTS THEREOF

The present invention is ideal for treatment of certain wounds. In a preferred embodiment, this method of treatment stimulates cell proliferation thus enhancing wound healing.

Wound healing is divided into three sequential, yet overlapping, phases: the inflammatory phase, the proliferative phase and the remodeling phase.

The inflammatory phase. The bleeding of the wound initiates thrombocyte aggregation at the injury site to form a coagel comprised of fibrin and thrombocytes, in order to stop the bleeding and also to protect the wound from bacterial contamination and fluid loss. Bacteria, dead tissue and foreign substances are phagocytosed and removed by macrophages. During this phase the formation of a provisional extracellular matrix take place, comprising fibrins, fibronectins and glycosaminoglycans. Inflammation proceeds until the wound is cleaned from unwanted substances and microorganisms.

The proliferative phase. Macrophages and thrombocytes are important factors in the proliferative phase as well due to the production and secretion of growth factors. The growth factors stimulate proliferation of endothelial cells, fibroblasts and smooth muscle cells. These cells will eventually replace the inflammatory cells in the wound. In the angiogenesis in this phase, new blood vessels are formed that supply the wound. with oxygen. Fibroblasts grow and initiate collagen production, which leads to granulation tissue formation resulting in reproduction of dermis and subcutis. The epithelium as well begins to reproduce by proliferation of keratinocytes. During the proliferative phase the wound is gradually made smaller.

The remodeling phase. During the remodeling phase the primitive extracellular matrix is degraded and simultaneously remodeling of a permanent matrix made up of collagen, elastin and proteoglycans take place.

Some patients have reduced cell proliferation, due to e.g., chemotherapy, age or nutritional diseases. This reduced cell proliferation can cause impaired wound healing. The present invention stimulates cell proliferation and is therefore ideal for treatment of certain wounds, especially wounds with a delayed healing process, due to reduced cell proliferation, and wounds where a quick healing process is desirable.

Further, there are several in inventions enhancing wound healing by stimulation of cell proliferation. However, none of the following inventions utilize hydrophobic substances. U.S. Pat. No. 6.699,287 discloses a dermal scaffold with wound healing effect by constituting microenvironments suitable for migration and proliferation of fibroblasts and vascular cells surrounding the wound.

In U.S. Pat. No. 5,457,093, gel formulations are described containing polypeptide growth factors having human mitogenic or angiogenic activity used for wound healing.

Patent application EP1637145 discloses an invention related to a wound healing promoting material. This material utilizes blood cells having wound healing effects which can improve the growth of fibroblasts, and allow them to produce growth factors involved in wound healing and thereby promoting wound healing.

In U.S. Pat. No. 5,720,981, an invention is described that provides a composition capable of stimulating growth and regeneration of epidermal cells. The composition comprises an aqueous, cell-free extract derived from epidermal cells effective to inhibit fibroblast proliferation in the wound region. The principle of binding microorganisms with hydrophobic dressings discussed above is a modem and overall effective Method for anti-microbial wound healing, which is described in U.S. Pat. No. 4,617,326 and U.S. Patent application 2006/0129080.

However, none of the prior art describes the use of hydrophobic substances, e.g., dialkyl carbamoyl chloride or alkyl ketene dimer, optionally applied to a carrier to induce increased cell proliferation, including but not limited to the proliferation of fibroblasts and keratinocytes.

Surprisingly, we have discovered that hydrophobic substances, as for example dialkyl carbamoyl chloride or alkyl ketene dimer, stimulate cell proliferation, for example in wounds and may when applied to a carrier be used as wound dressings for improved wound healing. The present invention may be used to enhance the natural healing process in wounds. The invention disclosed herein is based on the unexpected finding that hydrophobic substances promote cell proliferation.

Other objects and features of the invention will be more fully apparent from the following examples and appended claims.

### EXAMPLE 1

### Culturing Fibroblasts

Commercially available human dermal fibroblasts (CCL-110, ATCC/LGC-Standards AB, Boras, Sweden) were cultured in a culture incubator at 37° C. in 5% CO₂ atmosphere (Forma Science; AB Ninolab), using Eage's minimal essential medium with Earle's salts (EMEM, Sigma-Aldrich), supplemented with 10% foetal calf serum (FCS; Sigma-Aldrich). All handling of cells were carried out aseptically in a laminar airflow bench (Holten 2448; Ninolab).

Fibroblasts were initiated in culture, as previously described by Falk P. Experimental Models of the Human Perioneal Environment: Effects of TGF-beta and Hyaluronan; University of Gothenburg, 2008.

Prior to the start of the experiments performed in example 2 and 3, cells were cultured to approximately 50% confluence and then preincubated for 24 hours in a medium containing 1% FCS only. Cell count was done prior to the start of the experiments of example 2 and 3 to verify that here was no difference in cell count between the different wells.

Throughout the examples 2 and 3 below the non-parametric Kruskal-Wallis test was used to detect overall differences. with the Mann Whitney U test performed between individual groups. A p-value <0.05 was considered statistically significant. All calculations were performed using StatView software (v5.0; Abacus Concepts).

### EXAMPLE 2

### Cell Proliferation of Fibroblasts Using Dialkyl Carbamoyl Chloride

A sodium salt (XTT-assay, Sigma, USA) was used to study the influence on cell proliferation caused by the dialkyl carbamoyl chloride treated wound dressing as described in Example 5. Depending on the intra cellular dehydrogenase activity of the mitochondria, this salt will cause a colour change. The colour change is directly proportional to the increased number of cells and hence also directly proportional to cell proliferation. The colour change was measured using a 96-well plate reader (VMaz Kinetic ELISA; Molecular Devices) and software was used to calculate the absorbance at 450 nm (Softmax Pro, Molecular Devices). Cells were distributed to wells containing dialkyl carbamoyl chloride treated wound dressing material and culture medium and 12 wells only containing culture medium. Sterile disposable tools of 5 mm (Disposable Biopsy Punsch, Miltex, USA) were used to punch suitable pieces of the sterile wound dressing, which then were placed into the wells.

During proliferation studies, a medium without phenol red wa used to reduce the red background signal in the XTT assay. Absorbance at 450 nm (absorbance maximum), 650 nm (background absorbance) was measured. A total of eight wells were used in each setup for proliferation: four wells containing fibroblasts in the presence of dialkyl carbamoyl chloride treated dressings placed in the medium, four plates serving as untreated control (the experiment was run twice).

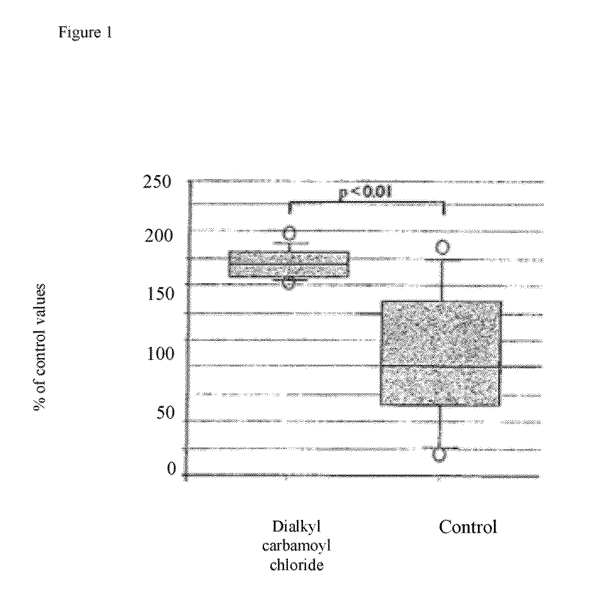

The results showed increased cell proliferation in wells containing the dialkyl carbamoyl chloride treated material compared to control wells. Absorbance in wells containing dialkyl carbamoyl chloride treated material is expressed as per cent of control values (background absorbance omitted). Dialkyl carbamoyl chloride induced cell proliferation even though no direct contact between the dialkyl carbamoyl chloride treated material and cells was obtained. Cell proliferation increased to 195% ±btcompared to 100%±1 m for untreated controls, shown in FIG. 1. Moreover the result shows that fibroblasts do riot bind to the dialkyl carbamoyl chloride treated material.

### EXAMPLE 3

### Dialkyl Carbamoyl Chloride Increases Cell Proliferation In Vivo

Twenty patients with wounds after transplantation of skin to another location are admitted to this open study. The patients are included in regularly planned follow ups with the medical staff. Inclusion criteria are the presence of a wound after skin has been removed for transplantation not more than 24 hours ago. The patients arc treated once daily using dressings manufactured according to example 3 herein. Ten of the admitted patients are treated with these cell proliferation inducing and ten patients serve as a control group treated with dressings without dialkyl carbamoyl chloride.

At the patient's first visit to the clinic, bedside assessment of the wounds is performed. and photos or the wound areas are taken. The dressing is applied by the investigator and the patients are carefully instructed how to do the application by themselves between the controls. The patients are admitted back to the clinic at day 5, 10 and 15 for bedside evaluation and photo documentation.

The wound healing assessment is made using Bates-Jensen wound assessment tool (BWAT) as a scoring system. BWAT is described by Carrie Sussman, Barbara M. Bates-Jensen (2007); Wound care: a collaborative practice manual (third edition) Lippincott Williams & Wilkins p. 176-179. In this study we examine the cell proliferation in wounds and the following evaluation is performed through photographs by a blinded independent observer according to protocol. Clinical and visual evaluation are done bedside and through photographs according to a preset evaluation protocol. All twenty patients complete the study. No side effects are noted.

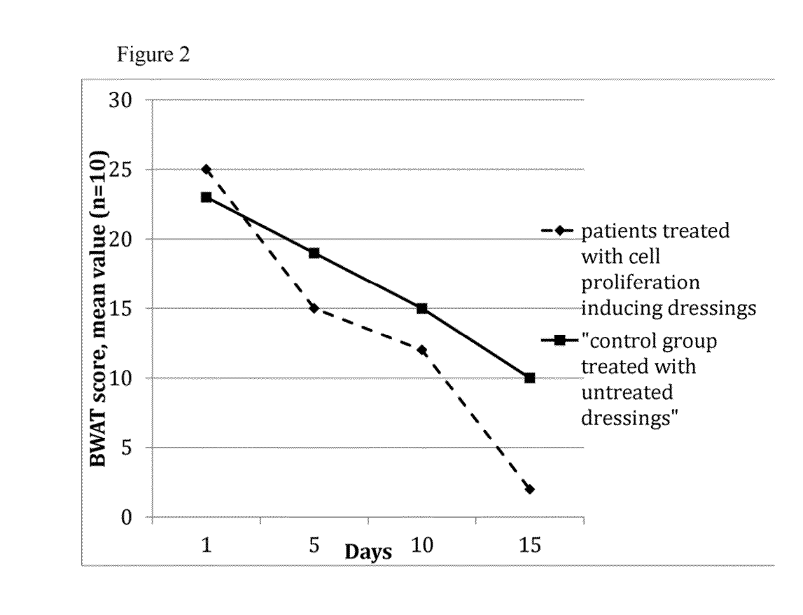

Pictures of the lesion are taken at day 1, 5, 10 and 15 by the investigator and blinded evaluation is performed by an independent investigator. The bedside assessments and the photo evaluation are used as basis for the BWAT scoring shown in FIG. 2. Patient treated with cell proliferation inducing dressings of Example 3 exhibits a more rapid wound healing process than the control group treated with dressings without dialkyl carbamoyl chloride.

### EXAMPLE 4

### Restoration of Damaged Fibroblast Surface

Fibroblasts cultured according to example 1 were grown until confluence at the start of the experiment. A mechanical injury was created by scraping the monolayer of cultured cells with a 10 μl sterile plastic pipette (D10ST, Gilson, INc.), forming a cross. The resulting damage was measured with a calibrated size marker at several time points, using a image processing software. In this in vitro model, with a monolayer of cells, cell proliferation and migration was identified by measuring the distance between the cells. To accomplish this, a line was drawn parallel to the wound edges, with multiple lines then drawn perpendicular to this, across the damaged area. This allowed a mean value for the length of the damaged area to be calculated. To reproduce measurements between incubation times, calculations were always made in the same the same area. Creating a cross in each culture well made it easy to identify this area by locating the centre of the cross and always beginning in the upper right arm of the cross.

Healing rate was calculated by dividing the restored distance by the time between each measurement. Measurements were performed at 1, 3, 6, 24, 32, 48, 56 and 72 hours, after creating the mechanical damage. In the wound-healing model, the compresses were cut into small pieces using the 5 mm biopsy punch (used in example 2). A total of 12 wells was used, with six wells used for the dialkyl carbamoyl chloride treated wound dressings as described in example 5 and six wells with medium only. Care was taken not to contaminate the culture wells with dressing debris or fibres.

Mechanical damage to the fibroblastic surface resulted in a denuded injury averaging a distance of 530±40 μm between the edges, with no significant difference between dialkyl carbamoyl chloride treated dressings and control wells (p=0.15). In all experiments using the dialkyl carbamoyl chloride dressing (n=12), the mechanical injury was fully recovered within 72 hours, while the cell culture wells with the control medium (n=12) failed to reach complete healing in the same period. The presence of the dressing caused the fibroblasts to migrate across the damaged area more rapidly than cells in medium only. This was illustrated with photomicrographs, with a greater number of fibroblasts repairing the denuded area in cells with the dialkyl carbamoyl chloride treated dressing, compared with cells treated with only the medium (results not shown).

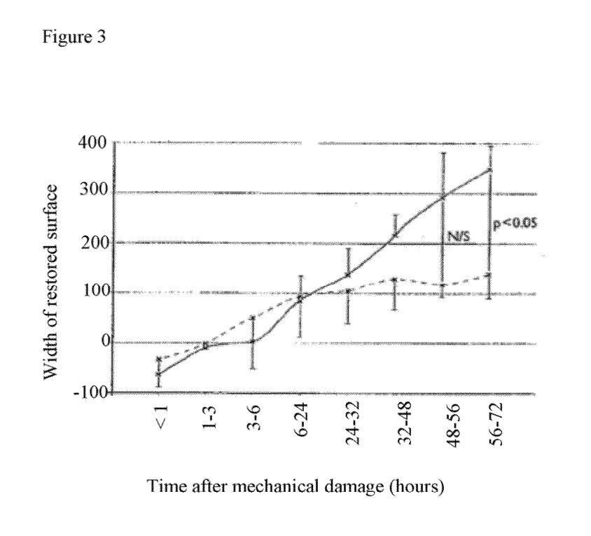

For total fibroblastic restoration, a significant difference between the dialkyl carbamoyl chloride treated dressing and the untreated control medium was observed between 56 and 72 hours (FIG. 3).

### EXAMPLE 4

### Manufacture of Wound Dressing Product

In this example we manufacture a standard wound dressing based on the invention in the following manner:

### Materials:

The hydrophobic layer is preferably produced by applying to a cellulose acetate fabric an amount of dialkyl carbamoyl chloride, making a covalent bond between the materials. The dialkyl carbamoyl chloride is applied by letting the fabric pass through a bath of 0.5-2% carbamoyl chloride solution. The applied amount is checked by using the hydrophobicity test below.

The acetate fabric is on rolls of 50 m length and at a width of 1 m, and taken as such to the next step.

### Hydrophobicity Test

### Equipment:

A fluid comprising formamide and ethylene glycol-monoethyl-ether with known dyne level between 37 and 50 dyne/cm.

Pipettes

Chronometer

This control is conducted in the beginning and the in the end of every roll. The test sample of acetate woven is 0.1 m².

Test method:

Alternative 1. The impregnated woven is stretched over a frame. Using the pipette a drop of liquid with a known dyne level is placed on the woven from approximately 5 mm's height. The chronometer is started. The drop shall stay on top of the woven for at least 30 seconds and if the drop is penetrating (flowing through) the woven, the test is reperformed with higher dyne level.

Alternative 2. This alternative is used when the woven material is sparse. Pour a fluid with known dyne level into a petri dish. Remove a fiber from the woven material and place it on the surface of the fluid. The fiber shall he floating during 30 seconds and if the fiber is sinking, the test needs to be reperformed with a fluid with higher dyne level.

Documentation. The result is documented for the batch in question.
Results. The sample shall have a dyne level less than 42 dyne/cm
Cutting and adding of carrier layer. The now bonded hydrophobic layer is cut into suitable size pieces for the final product, in this case 30 mm×30 mm. The cut pieces are placed on carrier-film, prepared with the adhesive and release paper and in strips of 100 mm. The bonded pieces are centered on the carrier strips and with a distance from center to center of 80 mm. A release paper of the same width is finally then applied on inner side. The now completely assembled dressing, is cut into pieces of 80 mm×100 mm, sterilized and then packed 10 by 10 in cartons.

What is claimed is:

1. A method for enhancing proliferation of cells, comprising bringing cells into contact with a hydrophobic substance.

2. The method according to claim 1, wherein said method is for use in healing of wounds.

3. The method according to claim 1, wherein said hydrophobic substance is selected from the group consisting of dialkyl carbamoyl and alkyl ketene dimer.

4. The method according to claim 2, wherein said hydrophobic substance is selected from the group consisting of dialkyl carbamoyl and alkyl ketene dimer and pharmaceutically acceptable salts and esters thereof.

5. The method according to claim 3, wherein said hydrophobic substance is a halide salt of dialkyl carbamoyl, preferably dialkyl carbamoyl chloride.

6. The method according to claim 4, wherein said hydrophobic substance is a halide salt of dialkyl carbamoyl, preferably dialkyl carbamoyl chloride.

7. A composition for use in enhancing proliferation of cells, comprising a hydrophobic substance, and optionally pharmaceutically acceptable carriers and excipients.

8. The composition according to claim 7, wherein said composition is a dressing.

9. The composition according to claim 8, for use in wound healing.

10. The composition according to claim 7, wherein said hydrophobic substance is selected from dialkyl carbamoyl and alkyl ketene dimer, and pharmaceutically acceptable salts and esters thereof.

11. The composition according to claim 8, wherein said hydrophobic substance is selected from dialkyl carbamoyl and alkyl ketene dimer, and pharmaceutically acceptable salts and esters thereof.

12. The composition according to claim 9, wherein said hydrophobic substance is selected from dialkyl carbamoyl and alkyl ketene dimer, and pharmaceutically acceptable salts and esters thereof.

13. The composition according to claim 7, wherein said hydrophobic substance is a halide salt of dialkyl carbamoyl, preferably dialkyl carbamoyl chloride.

14. The composition according to claim 8, wherein said hydrophobic substance is a halide salt of dialkyl carbamoyl, preferably dialkyl carbamoyl chloride.

15. The composition according to claim 9, wherein said hydrophobic substance s a halide salt of dialkyl carbamoyl, preferably dialkyl carbamoyl chloride.

16. The composition according to claim 10, wherein said hydrophobic substance is a halide salt of dialkyl carbamoyl, preferably carbamoyl chloride.

17. The composition according to claim 11, wherein said hydrophobic substance is a halide salt of dialkyl carbamoyl, preferably dialkyl carbamoyl chloride,

18. The composition according to claim 12, wherein said hydrophobic substance is a halide salt of dialkyl carbamoyl, preferably dialkyl carbamoyl chloride.

19. The composition according to claim 7, wherein the cells are selected from fibroblasts and keratinocytes.
